(19) United States
(12) Patent Application Publication
(10) Pub. No.: US 2003/0187455 A1
(43) Pub. Date: Oct. 2, 2003

(54) Intraocular lens insertion device

(76) Inventors: Kenichi Kobayashi, Tokyo (JP); Toshikazu Kikuchi, Tokyo (JP)

Correspondence Address: ROTH & GOLDMAN, P.A., 523 W. 6TH STREET, SUITE 707, LOS ANGELES, CA, 90014, US

(21) Appl. No.: 10/176,013

(22) Filed: Jun. 20, 2002

(30) Foreign Application Priority Data
Apr. 1, 2002 (JP) 2002-98948

(51) Int. Cl. A61F 9/00

(52) U.S. Cl. 606/107

(57) ABSTRACT

An intraocular lens insertion device for inserting an intraocular lens into an eye has a data display section. The data display section displays data regarding a lens loaded onto the intraocular lens insertion device. The data display section is provided at a predetermined visible position on an outer surface of the intraocular lens insertion device so as to enable the data regarding the loaded lens to be checked from the outside of the intraocular lens insertion device.

### BACKGROUND OF THE INVENTION

1. Field of the Invention

The present invention relates to an insertion device for inserting an intraocular lens into the eye, and more particularly to an intraocular lens insertion device which enables easy checking, from the outside of the insertion device, data, such as optical characteristics, of an intraocular lens placed in the insertion device.

2. Description of the Related Art

Implantation of an intraocular lens for treating cataract has been widely performed since an artificial lens; i.e., an intraocular lens, was implanted for the first time into the human eye in place of an opaqued natural lens during cataract surgery. The applicant of the present application has proposed various types of intraocular lens insertion devices for implanting an intraocular lens into the lenticular capsule from which an opaqued natural lens has been removed, as disclosed in Japanese Patent Application Laid-Open (kokai) Nos. 4-212350, 5-103803, 5-103808, 5-103809, and 7-23990. By use of these tools, the optical portion of an intraocular lens is compressed, rolled, bent, stretched, or folded so as to reduce its exterior size, thereby making it possible to insert the intraocular lens into the eye through a small incision formed in the eyeball. These insertion tools facilitate an operation for reliably implanting an intraocular lens into the eye.

FIG. 4A is a partially cutaway overall perspective view of a conventional insertion device for inserting a deformable intraocular lens; and FIG. 4B is an enlarged perspective view of a main portion of the insertion device.

In FIGS. 4A and 4B, reference numeral 11 denotes the insertion device for deforming an intraocular lens 10 to a smaller side and inserting the same into the eye; 12 denotes a hollow cylindrical device body; 13 denotes a screw sleeve; 14 denotes a push rod; 15 denotes an enclosing member having a lens receiving section 15a and an open/close mechanism 15b and adapted to deform the intraocular lens 10 to a smaller size; 16 denotes a slide stopper which engages the open/close mechanism 15b so as to maintain the same in a closed state; and 17 denotes an insertion tube.

When the insertion device 11 is used to insert the deformable intraocular lens 10 into the eye through a small incision, the open/close mechanism 15b of the enclosing member 15, which is connected to the front end 12a of the device body 12, is first opened. Subsequently, the deformable intraocular lens 10 is placed on the lens receiving section 15a of the enclosing member 15, and the open/close mechanism 15b of the enclosing member 15 is closed so as to reduce the exterior size of the deformable intraocular lens 10. Subsequently, the slide stopper 16 attached to the device body 12 is moved toward the lens receiving section 15a to engage the open/close mechanism 15b and bring the same in a closed state. Thus, placement of the intraocular lens 10 into the lens receiving section 15a is completed.

Subsequently, the screw sleeve 13, disposed at the rear of the device body 12, is moved toward the device body 12, brought into engagement with a screw portion 12b formed on the device body 12, and rotated in order to advance the push rod 14 in order to push forward the deformable intraocular lens 10 from the lens receiving section 15a. As a result, the deformable intraocular lens 10 is inserted into the eye through a tapered tip end 17a of an insertion tube 17 provided at the front end of the lens receiving section 15a, which tip end 17a has been inserted into the eye through a small incision formed in the eyeball. The operation of placing the intraocular lens 10 on the enclosing member 15 provided on the device body 12 is performed at the beginning of a surgical operation. Specifically, an intraocular lens 10 stored in an intraocular lens storage container 18 as shown in FIG. 5 is selected, removed from the container 18, and placed on the enclosing member 15.

Since eyesight differs among users (patients) who require intraocular lenses 10, many types of intraocular lens 10 are provided.

Therefore, in order to enable identification of a required intraocular lens 10 among many types, as shown in FIG. 5, lens data 18b are conventionally provided on a cover top face 18a of the storage container 18 storing an intraocular lens 10. The lens data include the name of a manufacturer of the lens, the model number of the lens, and other information. On the basis of the lens data, an operator selects a necessary intraocular lens 10, and sets the same onto the intraocular lens insertion device 11 for use at the time of an operation.

When operations are performed concurrently on a large number of users (patients), in some cases, one assistant, such as a nurse, sets different types of intraocular lenses 10 having necessary optical characteristics onto different intraocular lens insertion devices 11 to be used for the patients, and hands the intraocular lens insertion devices to an operating surgeon. In such a case, the operating surgeon cannot confirm the type of the intraocular lens 10 before inserting the same into the eye of a patient, and may erroneously insert into the eye of the patient an intraocular lens having optical characteristics that are not suitable for the patient.

### SUMMARY OF THE INVENTION

The present invention has been accomplished in order to solve the above-described problem involved in conventional intraocular lens insertion devices, and an object of the present invention is to provide an intraocular lens insertion device which can be manufactured at a plant in the form of a lens-containing insertion device loaded with an intraocular lens and which enables an user to know, from the outside of the insertion device, lens data, such as specifications and performance of the loaded intraocular lens.

Another object of the present invention is to provide an intraocular lens insertion device which enables an operating surgeon to confirm optical characteristics of an intraocular lens immediately before insertion thereof, even when an ordinary intraocular lens insertion device is used and the operating surgeon does not load the intraocular lens onto an intraocular lens insertion device (i.e., a nurse or assistant loads the intraocular lens onto an intraocular lens insertion device).

In order to achieve the above objects, the present invention provides an intraocular lens insertion device for inserting an intraocular lens into an eye, wherein data display means which displays data regarding a lens loaded onto the intraocular lens insertion device is provided at a predetermined visible position on an outer surface of the intraocular lens insertion device so as to enable the data regarding the loaded lens to be checked from the outside of the intraocular lens insertion device.

The present invention enables provision of an intraocular lens insertion device which can be manufactured at a plant in the form of a lens-containing insertion device loaded with an intraocular lens and which enables an user to know, from the outside of the insertion device, lens data, such as specifications and performance of an intraocular lens loaded onto the insertion device.

In addition, the present invention can be applied to the case in which a conventional insertion device is used; i.e., the case in which an intraocular lens is removed from a lens storage container and placed on an insertion device immediately before an operation. In this case, a decal on which data of the intraocular lens is printed is stored in the lens storage container together with the intraocular lens; and simultaneously with loading of the intraocular lens onto the insertion device, the decal is attached to a visible position of the outer surface of the device body. This enables an operating surgeon to confirm optical characteristics of an intraocular lens immediately before insertion thereof, even when the operating surgeon does not load the intraocular lens onto an intraocular lens insertion device.

Moreover, the insertion device having the above-described configuration enables an operator to check data of a lens loaded onto the insertion device on the basis of the data display means provided on the insertion device. Therefore, through employment of a system in which two persons (e.g., a nurse and an operating surgeon) check optical characteristics of an intraocular lens to be used in an operation, use of a wrong intraocular lens can be prevented, and thus safety of operation can be secured.

The data display means may assume any of the following forms:

a set of character data;

a change in color provided at a predetermined location on a device body of the intraocular lens insertion device;

a string of characters engraved, by use of a laser, at an externally visible location on the outer surface of the device body of the intraocular lens insertion device;a printed decal which carries character data and is visibly attached to the outer surface of the device body of the intraocular lens insertion device;

a string of characters or marks formed at an externally visible position on the device body of the intraocular lens insertion device during a molding process for molding the device body; and

a string of characters printed directly on the outer surface of the device body of the insertion device.

In the intraocular lens insertion device of the present invention, since character data are provided at a visible position on the outer surface of the insertion device by means of printing, engraving, or molding, a large amount of information can be displayed clearly, and checking of the character data is facilitated.

### BRIEF DESCRIPTION OF THE DRAWINGS

Various other objects, features and many of the attendant advantages of the present invention will be readily appreciated as the same becomes better understood by reference to the following detailed description of the preferred embodiment when considered in connection with the accompanying drawings, in which:

FIG. 1 is a front view of an intraocular lens insertion device according to an embodiment of the present invention;

FIG. 2 is a bottom view of the intraocular lens insertion device of FIG. 1;

FIGS. 3A to 3D are view showing a lens case wherein FIG. 3A is a plan view of the lens case, FIG. 3B is a left side view of the lens case, FIG. 3C is a right side view of the lens case, and FIG. 3D is cross-sectional view taken along line IIID-IIID of FIG. 3A;

FIGS. 4A and 4B are view showing a conventional intraocular lens insertion device, wherein FIG. 4A is an overall perspective view of the insertion device, and FIG. 4B is a perspective view of the lens enclosing member; and

FIG. 5 is a perspective view of a conventional storage container for storing an intraocular lens.

### DETAILED DESCRIPTION OF PREFERRED EMBODIMENT

An embodiment of the present invention will be described with reference to the accompanying drawings.

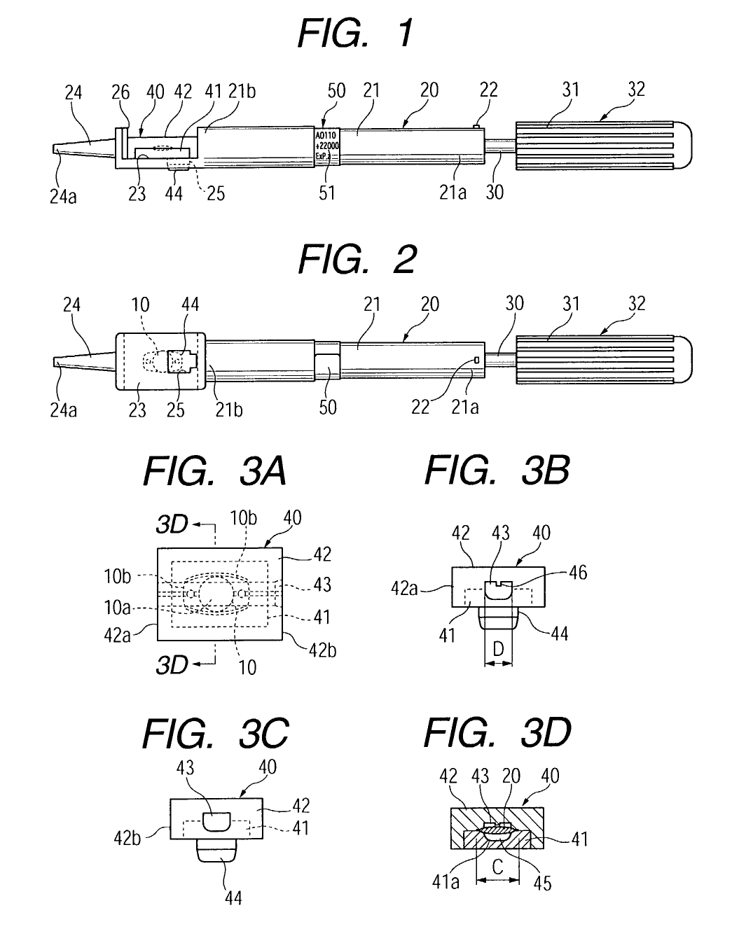

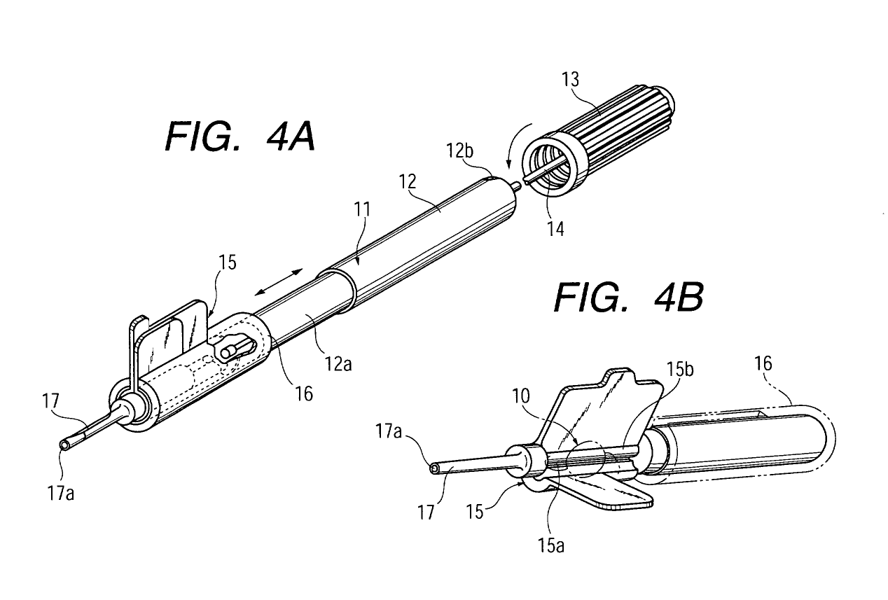

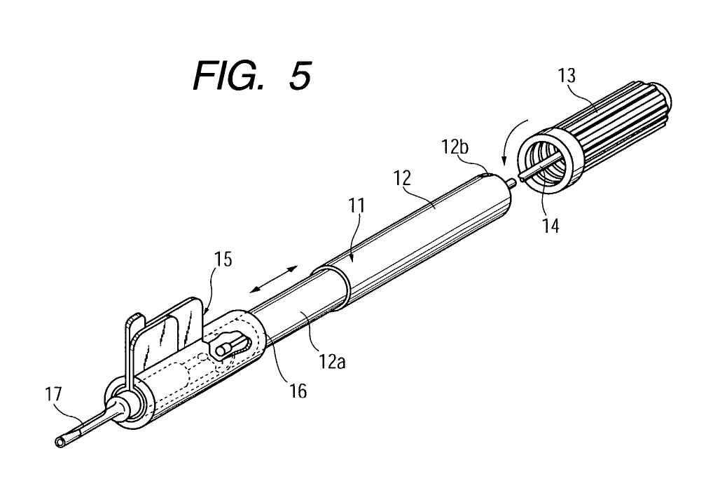

FIG. 1 is a front view of an intraocular lens insertion device according to an embodiment of the present invention, showing a state in which a lens case loaded with an intraocular lens is loaded onto the insertion device; and FIG. 2 is a bottom view of the device.

In FIGS. 1 and 2, reference numeral 20 denotes an intraocular lens insertion device which includes a hollow cylindrical device body 21, a push rod 30, a pusher mechanism 32, an attachment section 23, and a tapered insertion tube 24 and is adapted to deform an intraocular lens 10 to a smaller size and push out the same from a tip end portion 24a of the insertion tube 24. The device body 21 is formed of transparent or semi-transparent plastic and has a base end portion 21a of smaller diameter and a tip end portion 21b of larger diameter. The push rod 30 is disposed to be located on the center axis of the device body 21. The pusher mechanism 32 is constituted by a female screw sleeve 31, which is attached to a rear end portion of the push rod 30 and brought into screw-engagement with a male screw 22 formed on the base end portion 21a of the device body 21. With this configuration, the pusher mechanism 32 moves the push rod 30 back and forth upon rotation of the screw sleeve 31. The attachment portion 23 is formed on the tip end portion 21b and adapted to receive a lens case 40. The insertion tube 24 is formed on the tip end side of the attachment portion 23 and has a through-hole aligned with the center axis of the device body 21.

Reference numeral 25 denotes an engagement hole which is formed in the horizontal region of the attachment portion 23; and 26 denotes a notch which is formed on the rearward-facing surface of a front-side vertical wall of the attachment portion 23. The engagement hole 25 and the notch 26 cooperate to position the lens case 40 in relation to the attachment portion 23.

As is clearly shown in FIGS. 3A to 3D, the lens case 40 is constituted through assembly of a lens case top 41, on which the intraocular lens 10 is placed, and a lens case bottom 42 formed to cover the upper face of the intraocular lens 10 placed on the lens case top 41.

FIG. 3A is a plan view of the lens case, FIG. 3B is a left side view of the lens case, FIG. 3C is a right side view of the lens case, and FIG. 3D is a cross section taken along line 3D-3D in FIG. 3A.

A groove-shaped depression 41a is formed on the upper surface of the lens case top 41 such that the depression 41a extends in the horizontal direction in FIG. 3A. Support portions 10b of the intraocular lens 10 for supporting the optical portion 10a of the intraocular lens 10 are placed in the depression 41a. A projection 44 to be engaged with the engagement hole 25 of the attachment portion 23 is formed on the lower surface of the lens case top 41 to be located at a position which is offset slightly rightward from the center in FIG. 3A.

The lens case bottom 42 for covering the upper surface of the lens case top 41 is formed such that the lens case bottom 42 can be fitted onto the lens case top 41 while covering the upper surface and circumferential (side) surface of the lens case top 41. A through hole 43 is formed in the left and right walls 42a and 42b of the lens case bottom 42 such that the center axis of the through hole 43 is aligned with the center axis of the device body 21 of the insertion device 20 when the lens case 40 is attached to the attachment portion 23.

The specific procedure for placement of the lens case 40 is as follows. First, the intraocular lens 10 is placed on the lens case top 41 such that the optical portion 10a of the lens 10 corresponds to the depression 41a of the lens case top 41 and the support portions 10b of the intraocular lens 10 are supported by the edge portions of the depression 41a. Subsequently, the lens case bottom 42 is placed on the lens case top 41, so that a space 45 is formed between the lens case bottom 42 and the lens case top 41. The space 45 has a shape and size such that movement of the intraocular lens 10 within the space 45 is restricted. Further, the shape of the space 45 is determined such that the optical portion 10a of the intraocular lens 10 does not come into contact with the inner surface of the lens case top 41 or the inner surface of the lens case bottom 42, thereby preventing the optical characteristics of the optical portion 10a from changing due to force acting on the optical portion 10a during long-term storage.

The through hole 43 of the lens case 40 is reduced in diameter toward the tip end thereof. The intraocular lens 10 and the push rod 30 pass through the through hole 43. After attachment of the lens case 40 to the insertion device 20, the push rod 30 is advanced through operation of the pusher mechanism 32. As a result, the intraocular lens 10 within the lens case 40 is gradually deformed to a smaller size and moved into the insertion tube 24. Subsequently, after the tip end 24a of the insertion tube 24 is inserted into the eye through an incision formed therein, the push rod 30 is advanced further, so that the intraocular lens 10 is deformed to a further reduced size by means of the insertion tube 24 and then pushed into the eye.

FIGS. 3B, 3C, and 3D show the diameter of the through hole 43 of the lens case 40 being gradually reduced toward the tip end thereof. That is, the transverse dimension C of the space 45—which receives the intraocular lens 10 placed on the lens top case 41 of the lens case 40 as shown in FIG. 3D—is reduced to a transverse dimension D which corresponds to that measured at the tip end of the through hole 43 as shown in FIG. 3B. This configuration enables the intraocular lens 10 to enter a deformed state from a non-deformed state while being moved through the through hole 43 by means of the push rod 30. At the tip end, the through hole 43 has an asymmetrical shape with respect to the vertical direction. Further, a rail 46 projecting toward the center of the through hole 43 is formed such that the rail 46 extends in the direction of movement of the intraocular lens 10. This configuration enables the intraocular lens 10 to be formed into an intended shape.

Moreover, data display means 50 is provided on the outer surface of the device body 21 in order to visually display data, such as optical characteristics, of the intraocular lens 10.

Specifically, a sterilized, printed label serving as the display means 50 is bonded to the outer surface of the device body 21 in the vicinity of the attachment portion 23 to which the lens case 40 is attached. Data 51, such as optical characteristics, of the intraocular lens 10 placed within the lens case 40 are printed on the label.

When the intraocular lens insertion device according the present embodiment having the above-described configuration is manufactured at a plant, an intraocular lens is loaded onto the insertion device in order to obtain a lens-containing insertion device; and a printed label carrying data of the loaded intraocular lens, such as performance and specifications of the intraocular lens, is bonded to the device body of the insertion device.

In the intraocular lens insertion device of the present embodiment, the data display means 50 for displaying data 51, such as optical characteristics, of the intraocular lens 10 placed within the lens case 40 of the insertion device 20 is provided at a visible position on the outer surface of the device body 21 of the intraocular lens insertion device 20. Therefore, even when the intraocular lens insertion device 20 is shipped from a plant in the form of a lens-containing insertion device onto which a new intraocular lens 10 has been loaded, the data 51, such as optical characteristics, of the intraocular lens 10 within the insertion device 20 can be confirmed with ease immediately before an operation, to thereby prevent an improper operation such as use of a wrong intraocular lens.

Examples of the data 51 regarding the intraocular lens 10 include the name of a manufacture of the lens, the model name of the lens, the power of the lens, an effective sterilization period, information regarding a manner of use (fixing position), information regarding use of the lens such as matters to be attended to, the manufacturing date of the lens, a serial number, and a model number.

The data display means 50 may assume any of the following forms:

(1) a set of character data;

(2) a change in color provided at a predetermined location on the device body 21 of the intraocular lens insertion device 20;

(3) a string of characters engraved, by use of a laser, at an externally visible location on the outer surface of the device body 21 of the intraocular lens insertion device 20;

(4) a printed decal which carries character data and is visibly attached to the outer surface of the device body 21 of the intraocular lens insertion device 20; and

(5) a string of characters or marks formed at an externally visible position on the device body 21 of the intraocular lens insertion device 20 during a molding process for molding the device body 21.

Moreover, the lens data can be displayed not only by means of characters, but also by means of lines, dots, colors, or code.

In the above-described embodiment, the intraocular lens insertion device 20 has been described as being shipped from a plant in the form of a lens-containing insertion device loaded with an intraocular lens 10. However, the present invention is not limited thereto, and can be applied to the case in which a conventional insertion device is used; i.e., the case in which an intraocular lens 10 is removed from a lens storage container 18 and placed on an insertion device 20 immediately before an operation. In this case, a decal on which data of the intraocular lens 10 is printed is stored in the lens storage container 18 together with the intraocular lens 10; and simultaneously with loading of the intraocular lens 10 onto the insertion device 20, the decal is attached to a visible position of the outer surface of the device body. This enables an operating surgeon to confirm optical characteristics of an intraocular lens immediately before insertion thereof, even when the operating surgeon does not load the intraocular lens onto an intraocular lens insertion device.

In addition, even in the case in which insertion of an intraocular lens is performed by use of an operation tool, such as tweezers (not shown), other than the insertion tool 20 having a tapered insertion tube, data regarding the intraocular lens can be confirmed through employment of a lens storage container 18 which contains a decal on which data of the intraocular lens 10 have been printed or an insertion device on which data of the intraocular lens 10 have been imprinted.

Obviously, numerous modifications and variations of the present invention are possible in light of the above teachings. It is therefore to be understood that within the scope of the appended claims, the present invention may be practiced otherwise than as specifically described herein.

What is claimed is:

1. An intraocular lens insertion device for inserting an intraocular lens into an eye, comprising:
data display means which displays data regarding a lens loaded onto the intraocular lens insertion device, the data display means being provided at a predetermined visible position on an outer surface of the intraocular lens insertion device so as to enable the data regarding the loaded lens to be checked from the outside of the intraocular lens insertion device.

2. An intraocular lens insertion device according to claim 1, wherein the data display means is a set of character data.

3. An intraocular lens insertion device according to claim 1, wherein the data display means is a change in color provided at a predetermined location on a device body of the intraocular lens insertion device.

4. An intraocular lens insertion device according to claim 1, wherein the data display means is a string of characters engraved, by use of a laser, at an externally visible location on an outer surface of a device body of the intraocular lens insertion device.

5. An intraocular lens insertion device according to claim 1, wherein the data display means is a printed decal which carries character data and is visibly attached to an outer surface of a device body of the intraocular lens insertion device.

6. An intraocular lens insertion device according to claim 1, wherein the data display means is a string of characters at an externally visible position on a device body of the intraocular lens insertion device during a molding process for molding the device body.

7. An intraocular lens insertion device according to claim 1, wherein the data display means is a string of characters printed directly on an outer surface of a device body of the intraocular lens insertion device.

8. An intraocular lens insertion device according to claim 2, wherein the data display means is a string of characters engraved, by use of a laser, at an externally visible location on an outer surface of a device body of the intraocular lens insertion device.

9. An intraocular lens insertion device according to claim 2, wherein the data display means is a printed decal which carries character data and is visibly attached to an outer surface of a device body of the intraocular lens insertion device.

10. An intraocular lens insertion device according to claim 2, wherein the data display means is a string of characters at an externally visible position on a device body of the intraocular lens insertion device during a molding process for molding the device body.

11. An intraocular lens insertion device according to claim 2, wherein the data display means is a string of characters printed directly on an outer surface of a device body of the intraocular lens insertion device.
